(19) United States
(12) Patent Application Publication
(10) Pub. No.: US 2018/0116531 A1
(43) Pub. Date: May 3, 2018

(54) PULSEBEAT MEASUREMENT APPARATUS, WEARABLE DEVICE AND PULSEBEAT MEASUREMENT METHOD

(71) Applicant: KDDI CORPORATION, Tokyo (JP)

(72) Inventors: Masahiro Suzuki, Fujimino-shi (JP); Tomoaki Ueda, Tokyo (JP)

(21) Appl. No.: 15/855,674

(22) Filed: Dec. 27, 2017

Related U.S. Application Data
(63) Continuation of application No. PCT/JP2016/068507, filed on Jun. 22, 2016.

(30) Foreign Application Priority Data
Jul. 9, 2015 (JP) 2015-137571
Aug. 3, 2015 (JP) 2015-153156

(51) Int. Cl. A61B 5/0205; A61B 5/00

(52) U.S. Cl. CPC A61B 5/02055; A61B 5/7207; A61B 5/002; A61B 5/0456; A61B 5/7435; A61B 5/6803; A61B 5/681

(57) ABSTRACT

A pulsebeat measurement apparatus includes: a temperature measurement unit configured to measure, by contacting a human body, a temperature of a contact surface; and a processing unit configured to process a measurement result obtained by the temperature measurement unit. The processing unit includes: an extraction unit configured to extract a change in temperature caused by a pulsation based on the measurement result, and a pulsebeat measurement unit configured to measure a pulsebeat based on an interval between the changes in temperature.

This application is a continuation of International Patent Application No. PCT/JP2016/068507 filed on Jun. 22, 2016, and claims priority to Japanese Patent Applications No. 2015-137571 filed on Jul. 9, 2015 and No. 2015-153156 filed on Aug. 3, 2015, the entire contents of which are incorporated herein by reference.

### TECHNICAL FIELD

The present invention relates to a pulsebeat or heartbeat measurement apparatus for measuring the pulsebeat of a user, a wearable device, and a pulsebeat measurement method.

### BACKGROUND ART

In recent years, a computer such as a wristwatch, ring, or a pair of glasses which can be directly worn and carried by the user (so-called wearable device) is attracting attention. Since there is no big difference between simply wearing and carrying a small computer, an application technique which makes the best use of a feature of always wearing is required for the wearable device. As such application technique, a vital sensing technique of automatically recording the condition of the user at the time of wearing is plausible. An example of the vital sensing technique is pulsebeat measurement.

In general, as pulsebeat measurement, there is known electrocardiography of detecting a heart rate almost equivalent to a pulsebeat rate using the peak of an electrocardiographic waveform measured by attaching electrodes to a living body, for example a P wave, an R wave, and the like. There is also known photoplethysmography of irradiating a peripheral blood vessel such as a wrist, finger, or earlobe with light, and detecting pulsebeat based on an optical change in which reflected light periodically changes due to a blood flow and light absorption characteristic.

“Regarding Development and Practical Use of “hitoe” Which Is a Functional Material That Enable Biometric Information to Be Continuous Measured Just by Wearing It”, Internet [URL: https://www.nttdocomo.co.jp/info/news_release/2014/01/3 0_00.html], <search on Jun. 5, 2015>, discloses an apparatus capable of performing heart beat measurement by embedding, in clothing, a measurement electrode according to a sport electrocardiographic lead system, and wearing it. Furthermore, Japanese Patent Laid-Open No. 2006-102161 discloses an arrangement of measuring a heart beat by wearing, on a pinna, an apparatus including a sensor for performing irradiation with an infrared ray.

### SUMMARY OF INVENTION

### Technical Problem

The arrangement disclosed in the above internet document can correctly measure a heart beat since the electrode is worn on the body surface. Since, however, it is necessary to bring the electrode into tight contact with the human body, he/she has an unwell feeling such as a restraint feeling or oppressive feeling. In addition, it is necessary to wash the clothing, and the washing count is limited in terms of durability, thereby impairing the usability. In the arrangement disclosed in Japanese Patent Laid-Open No. 2006-102161, the power consumption of a light emitting element is large. Therefore, for example, if the arrangement is used in a small terminal apparatus such as a wearable device, it is difficult to continuously measure a pulsebeat all the time. In addition, if the user has a tattoo or the like, a coloring matter blocks light, and thus it may be impossible to capture reflected light appropriately. Consequently, a pulsebeat measurement method using a new technique which puts no load on either the human body or the apparatus is desired.

### Solution to Problem

According to one aspect of the present invention, a pulsebeat measurement apparatus includes: a temperature measurement unit configured to measure, by contacting a human body, a temperature of a contact surface; and a processing unit configured to process a measurement result obtained by the temperature measurement unit. The processing unit includes: an extraction unit configured to extract a change in temperature caused by a pulsation based on the measurement result, and a pulsebeat measurement unit configured to measure a pulsebeat based on an interval between the changes in temperature.

Other features and advantages of the present invention will be apparent from the following description taken in conjunction with the accompanying drawings. Note that the same reference numerals denote the same or like components throughout the accompanying drawings.

### BRIEF DESCRIPTION OF DRAWINGS

FIG. 1 is a view showing an overview of a pulsebeat measurement method by a pulsebeat measurement apparatus according to an embodiment;

FIG. 2 is a block diagram showing the hardware arrangement of the pulsebeat measurement apparatus according to an embodiment;

FIG. 3 is a functional block diagram showing the pulsebeat measurement apparatus according to an embodiment;

FIG. 4 is a view showing an analog biological signal on which AC noise is superimposed according to an embodiment;

FIG. 5 is a view showing the mechanism of a FIFO memory according to an embodiment;

FIG. 6 is a view showing addition processing in a calculation unit according to an embodiment;

FIG. 7 is a view for explaining noise removal according to an embodiment;

FIG. 8 is a flowchart illustrating a pulsebeat measurement method according to an embodiment;

FIG. 9 shows an example of experiment data indicating the relationship between a body temperature and a pulsebeat;

FIG. 10 is a view showing a wearable device according to an embodiment;

FIG. 11 is a functional block diagram showing a pulsebeat measurement apparatus according to an embodiment;

FIG. 12 is a functional block diagram showing a pulsebeat measurement apparatus according to an embodiment;

FIG. 13 is a view showing processing in a combining unit according to an embodiment; and

FIG. 14 is a view showing a wearable device according to an embodiment.

### DESCRIPTION OF EMBODIMENTS

### First Embodiment

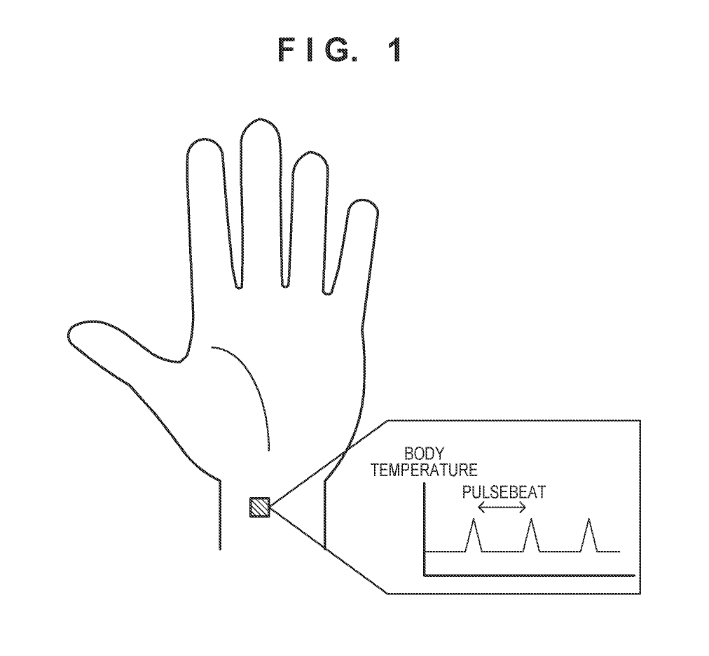

An outline of a pulsebeat measurement apparatus according to this embodiment will be described first with reference to FIG. 1. FIG. 1 is a view for explaining a pulsebeat measurement method by the pulsebeat measurement apparatus. As shown in FIG. 1, the pulsebeat measurement apparatus according to this embodiment detects small changes in body temperature in a human body portion (for example, a wrist, neck, ankle, or the like) where a blood vessel (artery) is close to the surface, and measures a pulsebeat based on the interval between the small changes in body temperature.

Conventionally, it is known that the body temperature of the human body changes due to an exercise, time (early morning, daytime, or the like), temperature, meal, sleep, female sexual cycle, emotion, and the like. In these cases, it is considered that the body temperature gradually rises or lowers, and never abruptly changes. In this regard, the present inventors observed a change in human body temperature using a temperature sensor with high sensitivity, and could confirm not only a gradual change in temperature in daily life but also a phenomenon in which the body temperature instantaneously rises or lowers in correlation with the pulsebeat. This phenomenon is estimated to occur when a situation in which blood warmed in the heart reaches a measurement portion to cause an instantaneous rise in temperature in the measurement portion, and is dissipated before a subsequent pulsation is repeated.

In this embodiment, a pulsebeat is measured by detecting an instantaneous small rise in temperature caused by a pulsation. At this time, since a change in temperature to be detected is small (for example, about 0.01° C. to 0.05° C.), it is readily influenced by noise. To solve this problem, this embodiment makes it possible to detect a small change in temperature by performing noise removal (to be described later).

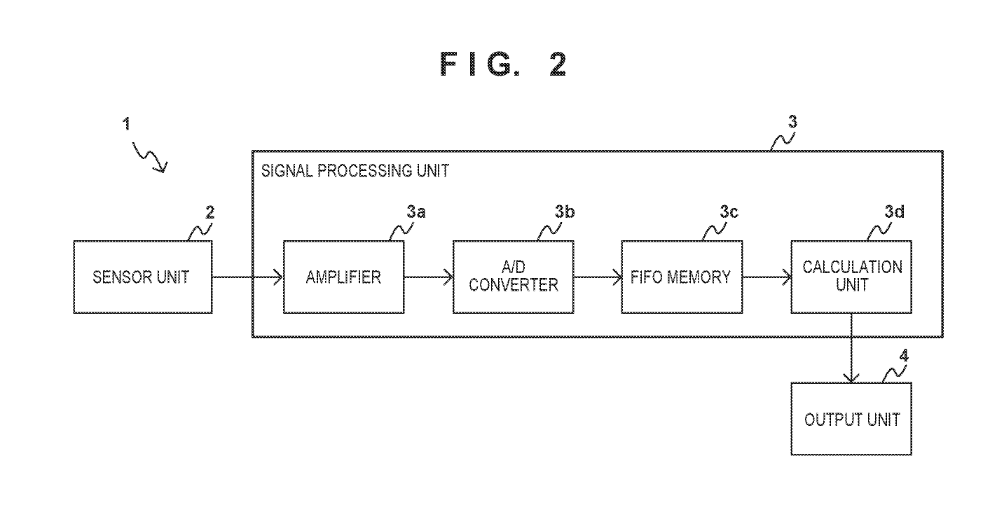

FIG. 2 is a block diagram showing the hardware arrangement of a pulsebeat measurement apparatus 1 according to this embodiment. The pulsebeat measurement apparatus 1 includes a sensor unit 2, a signal processing unit 3, and an output unit 4. The sensor unit 2 serves as a contact temperature sensor for measuring, by contacting a human body, the temperature of a contact surface. That is, the sensor unit 2 operates as a temperature measurement unit. Blood sent from the heart is warmed in the heart or the like, and lowers in temperature while circulating through the whole body. Therefore, when measuring a rise in temperature caused by a pulsation (blood), it is preferable to perform measurement on the upstream side (in the main artery) of the blood circulation path, and the sensor unit 2 is arranged near, for example, the main artery on the inner side of a wrist (on the palm side).

Note that the type of the sensor unit 2 is arbitrary, and a resistance temperature sensor such as a thermistor can be used based on the viewpoint of power saving and a low cost. The resistance temperature sensor measures a temperature by measuring the resistance of the sensor which changes in accordance the temperature, and only a small current (the order of about mA to μA) is necessary for resistance measurement. Therefore, it is possible to measure a pulsebeat with very small power, as compared with photoplethysmography which uses a light emitting element with large power consumption. A high-precision sensor such as a platinum thin film temperature sensor can also be used, as a matter of course.

As described above, in this embodiment, a pulsebeat is measured by detecting a small rise in body temperature. If a thermistor is used as the sensor unit 2, a temperature sensor having a small heat capacity can be used to react to such small change in temperature. To prevent heat (for example, the heat of the signal processing unit 3) other than body heat from being transferred to the sensor unit 2, a heat insulation unit for suppressing transfer of heat can be provided between the sensor unit 2 and the signal processing unit 3. Note that the pulsebeat measurement apparatus 1 can be provided with a heat dissipation unit (not shown), as needed. This heat dissipation unit absorbs or dissipates the heat of the sensor unit 2 which has risen in temperature in association with a pulsation, thereby keeping the temperature of the sensor unit 2 almost constant before or after the pulsation.

The signal processing unit 3 processes the measurement result of the sensor unit 2, that is, the temperature (resistance value) measured by the sensor unit 2, and measures a pulsebeat based on the interval between the timings at which the temperature rises in association with a pulsation. An amplifier 3a amplifies an analog biological signal (temperature data) input from the sensor unit 2, and outputs the amplified signal. Note that if it is not necessary to amplify the signal, the amplifier 3a is unnecessary. The amplification factor of the amplifier 3a is arbitrarily set, as needed. If, however, a thermistor is used as the sensor unit 2, commercial power supply noise may be superimposed on the biological signal measured by the sensor unit 2, and thus an amplification factor (for example, up to about 100) that prevents the amplified biological signal, on which noise is superimposed, from falling outside the input range of an A/D converter 3b is set.

The A/D converter 3b converts the analog biological signal output from the amplifier 3a into digital data (digital biological signals) at a predetermined sampling frequency. In general, the pulse rate of the human body is several Hz, and a band of several tens of Hz suffices for measurement for detecting a pulsebeat. Thus, a sampling frequency may be low. Based on the sampling theorem, the band is the half of the sampling frequency. Thus, a low sampling frequency also functions as a low-pass filter (LPF), and can remove unnecessary high-frequency noise at the time of conversion into digital data.

Note that since the sensor unit 2 uses a small sensing current for the purpose of power saving, a circuit such as a sensor may function as an antenna and may be influenced by leakage current noise (commercial power supply noise) from an electric wiring or high-voltage transmission line. Since the commercial power supply noise is periodic noise, noise for one period is averaged by adding positive and negative components, thereby obtaining zero or a constant value. That is, the commercial power supply noise can be readily removed by calculating a moving average for one period. Therefore, for example, a sampling frequency is set (to, for example, an integer multiple of one period of the commercial power supply noise) in accordance with the period of the commercial power supply noise so that the commercial power supply noise for one period is superimposed on predetermined sampling periods of the digital biological signals. This arrangement can readily remove the commercial power supply noise.

Based on the sampling theorem, frequency components falling outside the band width of ½ of the sampling frequency are represented as aliasing. This aliasing can be removed based on a cutoff frequency obtained by a moving average. At this time, if the frequency of the commercial power supply noise and the band where aliasing is generated are very different, they do not influence each other. By setting, as a sampling frequency, 800 Hz which is 16 times higher than the commercial power supply frequency (50 Hz), the band (400 Hz) where aliasing is generated can be largely differentiated from the frequency (50 Hz) of the commercial power supply noise, thereby removing the commercial power supply noise and aliasing. Note that if 800 Hz is set as a sampling frequency, the commercial power supply noise of 50 Hz for one period is superimposed on 16 sampling periods of the digital biological signals.

A FIFO memory 3c stores the digital biological signals converted into the digital data in the A/D converter 3b. The FIFO memory 3c is updated by sequentially storing, for each clock signal of a frequency which is equal to an integer multiple of the frequency of the commercial power supply noise, the divided digital biological signals for one period, the number of which is equal to the integer multiple. In this embodiment, since the frequency of the commercial power supply noise is 50 Hz and the sampling frequency is 800 Hz, a digital biological signal generated by sampling the analog biological signal is stored in the FIFO memory 3c 800 times per sec.

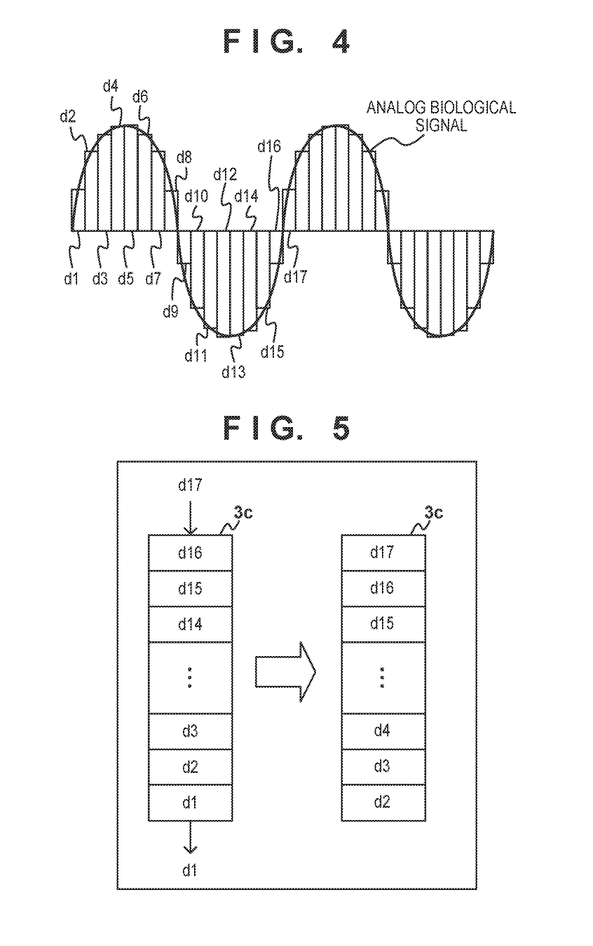

The FIFO memory 3c is a memory which accumulates a predetermined number of data for a predetermined time width, and from which data having arrived first is extracted after a predetermined time elapses. If new data is stored, old data is deleted. FIG. 4 shows the analog biological signal on which the commercial power supply noise is superimposed, and the digital biological signals (d1 to d17) obtained by sampling the analog biological signal in the A/D converter 3b.

The A/D converter 3b converts an analog biological signal into digital biological signals at a predetermined sampling frequency. Note that in the example shown in FIG. 4, the digital biological signals are obtained by sampling the analog biological signal for every ⅛ period of the commercial power supply noise. In addition, FIG. 4 shows an example in which the level of the commercial power supply noise is higher than that of the analog biological signal.

As shown in FIG. 5, the FIFO memory 3c sequentially stores the 16 digital biological signal data for one period of the commercial power supply noise. As shown in FIG. 5, if the A/D converter 3b outputs the digital biological signal d17 on which the commercial power supply noise is superimposed while the FIFO memory 3c stores the digital biological signals d1 to d16 on which the commercial power supply noise is superimposed, the FIFO memory 3c deletes the oldest digital biological signal d1 on which the commercial power supply noise is superimposed, and newly stores the digital biological signal d17 on which the commercial power supply noise is superimposed.

Referring back to FIG. 2, a calculation unit 3d obtains, at the commercial power supply frequency, the moving average of the digital biological signals for one period stored in the FIFO memory 3c, thereby removing noise such as commercial power supply noise. A moving average calculation method will now be described. A digital biological signal stored in the FIFO memory 3c is represented by do where n is an integer of 0 or more, and indicates the input order in the FIFO memory 3c. If the digital biological signals for one period are stored in the FIFO memory 3c, the calculation unit 3d calculates an addition result Sum0 by adding digital biological signals d0 to d15 stored in the FIFO memory 3c, and saves it.

If a new digital biological signal d16 is input to the FIFO memory 3c, the FIFO memory 3c outputs the digital biological signal d0, and stores the digital biological signal d16. If the FIFO memory 3c is updated, the calculation unit 3d calculates an addition result Sum1 by adding the digital biological signals d1 to d16 stored in the updated FIFO memory 3c, and saves it. In this way, every time a new digital biological signal is input to the FIFO memory 3c and the FIFO memory 3c is updated, the calculation unit 3d calculates an addition result Sumx (x=0, 1, . . . ), and saves it.

If, however, the digital biological signals stored in the FIFO memory 3c are added every time the FIFO memory 3c is updated, the calculation load unwantedly becomes heavy. To solve this problem, it is possible to adopt an arrangement of calculating an addition result after update based on the difference between the FIFO memory 3c before update and that after update and the previous addition result of the FIFO memory 3c.

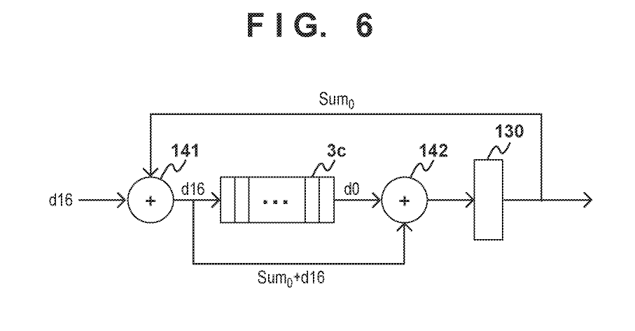

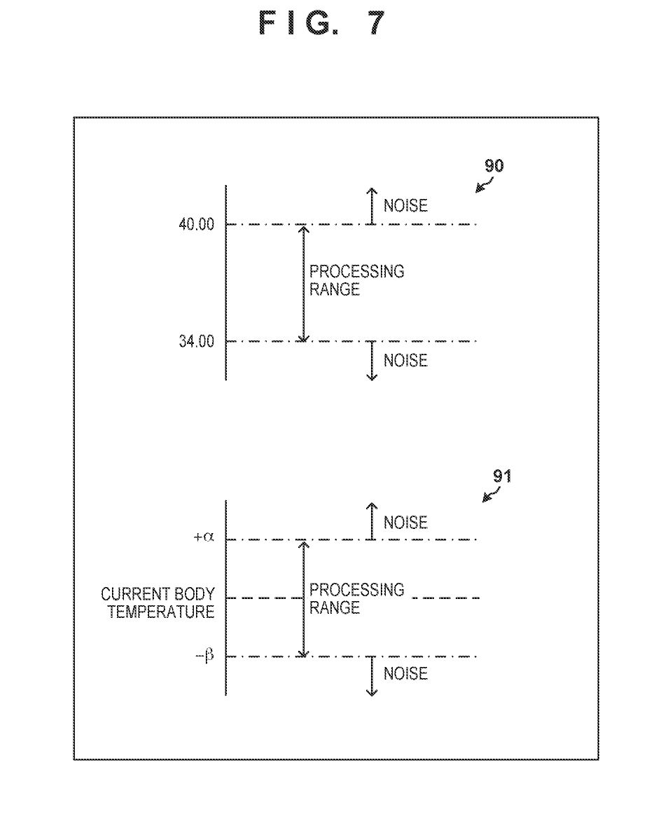

Practical processing will be described with reference to FIG. 6. Note that in FIG. 6, the FIFO memory 3c already stores the digital biological signals d0 to d15. If the FIFO memory 3c stores the digital biological signals d0 to d15, an addition result 130 stores the value Sum0 obtained by accumulating the digital biological signals d0 to d15. If the A/D converter 3b inputs the new digital biological signal d16, a calculator 141 adds the previous addition result Sum0 and the input digital biological signal d16. Next, if the digital biological signal d16 is input to the FIFO memory 3c, the FIFO memory 3c outputs the digital biological signal d0, and newly stores the digital biological signal d16. A calculator 142 subtracts, from (Sum0+d16) output from the calculator 141, the digital biological signal d0 output from the FIFO memory 3c, thereby calculating the addition result Sum1 after update.

The calculation unit 3d calculates a moving average by dividing the thus obtained addition result by the number of digital biological signals stored in the FIFO memory 3c, thereby removing noise contained in the digital biological signals. The moving average is a filter which averages n closest data and uses the average value as a representative value, and is a kind of low-pass filter. In this embodiment, the moving average of 16 points at a sampling frequency of 800 Hz is used, and a cutoff frequency is about 22 Hz (=0.443×800 Hz/16).

As described above, the commercial power supply noise as a sinusoidal wave can be removed by the moving average for one period. In addition, if the sampling frequency is 800 Hz, aliasing appears in a band of 400 Hz or more, and can thus be removed by the cutoff frequency associated with the moving average. In this case, since the band of 400 Hz or more of the aliasing and the frequency (50 Hz) of the commercial power supply noise are largely different from each other, they do not influence each other, and both the aliasing and the commercial power supply noise can be removed.

Note that in this embodiment, a ΣA A/D converter using the ΣA modulation method can be used as the A/D converter 3b. This is because, for example, if a flash A/D converter or successive approximation A/D converter is used, it has a quantization error, and thus noise may remain even after noise removal is performed. If a successive approximation A/D converter is used, quantization noise becomes 1/√n by addition of n signals, and thus noise remains. To the contrary, since a ΣA A/D converter has a feature in which a conversion cumulative error (the result of integration) is always smaller than 1, even if the same calculation is performed, quantization noise can be 1/n, thereby obtaining a satisfactory measurement waveform.

The calculation unit 3d performs peak detection processing for the digital biological signals (temperature data) from which noise has been removed, and detects, as a pulsation timing, a timing at which the temperature instantaneously, slightly rises. The calculation unit 3d calculates a pulse rate based on the interval (so-called R-R interval) between the timings at which the temperature rises in association with a pulsation.

The output unit 4 outputs the pulse rate measured by the signal processing unit 3. The output form from the output unit 4 is arbitrary. For example, the output unit 4 displays or prints the measured pulse rate, or transmits it to an external device.

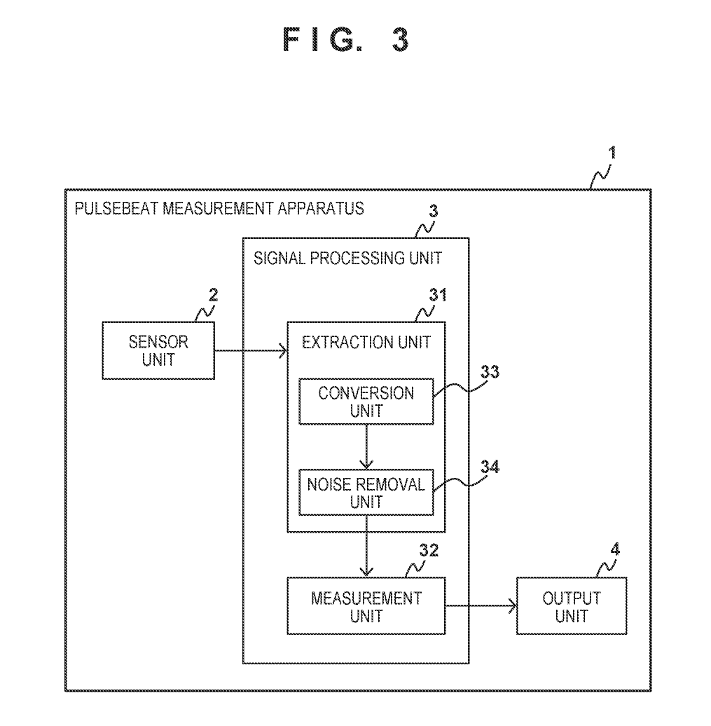

FIG. 3 is a functional block diagram showing the pulsebeat measurement apparatus 1. An extraction unit 31 is formed by including a conversion unit 33 and a noise removal unit 34 to extract a change in temperature caused by a pulsation from the temperature (analog data) measured by the sensor unit 2. The conversion unit 33 mainly corresponds to the amplifier 3a and A/D converter 3b of FIG. 2, and digitally converts the analog measurement result (analog biological signal) of the sensor unit 2 at a sampling frequency which is equal to an integer multiple of the frequency of noise to be removed. For example, since the commercial power supply frequency is 50 Hz in eastern Japan, the conversion unit 33 converts an analog biological signal into digital biological signals at a sampling frequency (800 Hz) which is 16 times higher than the frequency of the commercial power supply noise to be removed. On the other hand, since the commercial power supply frequency is 60 Hz in western Japan, the conversion unit 33 converts an analog biological signal into digital biological signals at a sampling frequency (780 Hz) which is 13 times higher than the frequency (60 Hz) of the commercial power supply noise. The sampling frequency may be set to 800 Hz (13.333 . . . times higher than the frequency of the commercial power supply noise) even in western Japan. In this case, noise is removed by calculating the moving average of 13 signals. Although it is impossible to completely remove the influence of the commercial power supply noise, it is possible to sufficiently detect a change in temperature caused by a pulsation. Note that the commercial power supply frequency to be removed may be manually switched (eastern Japan and western Japan may be manually switched). Alternatively, the commercial power supply frequency may be determined and switched by separating the sensor unit 2 and comparing only the noise levels. An arrangement of switching the commercial power supply frequency using positioning information of a GPS or the like may also be adopted.

The noise removal unit 34 mainly corresponds to the FIFO memory 3c and calculation unit 3d of FIG. 2, and performs noise removal for the measurement result of the sensor unit 2 by calculating the moving average of the digital measurement results (digital biological signals) converted by the conversion unit 33 using a number corresponding to the magnification between the sampling frequency and the noise frequency. More specifically, the noise removal unit 34 removes the commercial power supply noise as a sinusoidal wave by calculating the moving average and adding the positive and negative components, and also removes the aliasing by the cutoff frequency associated with the moving average.

As described above, if the moving average of 16 points is used at a sampling frequency of 800 Hz, the cutoff frequency is about 22 Hz. In this regard, the number of points of the moving average may be multiplied by an integer in accordance with the noise status. For example, by using the moving average of 32 points at a sampling frequency of 800 Hz, a cutoff frequency of about 11 Hz (=0.443×800 Hz/32) can be obtained. In this case as well, a frequency band necessary for pulsebeat measurement can be passed, thereby correctly measuring a pulsebeat.

Since the temperature of the human body is detected for pulsebeat measurement, a temperature falling outside a temperature range which the human body temperature can take can be removed as temperature noise. As shown in a graph 90 of FIG. 7, the noise removal unit 34 can deal with, as noise, a temperature falling outside a predetermined temperature range (for example, a range of 34° C. to 40° C.) which the human body temperature can take, among the temperatures measured by the sensor unit 2, and can remove the temperature from the processing target.

Since a change in temperature caused by a pulsation is small, a predetermined temperature range (for example, a range of about ±0.5° C.) with reference to the current body temperature measured by the sensor unit 2 may be set as a processing target, and a temperature falling outside the range may be dealt with as noise, as shown in a graph 91 of FIG. 7. This can further reduce noise. Note that since the body temperature rises at the time of a pulsation, the width from the current body temperature to the upper limit of the predetermined temperature range may be different from the width from the current body temperature to the lower limit of the predetermined temperature range (more specifically, the width from the current body temperature to the upper limit is made larger). Furthermore, since a rise in body temperature caused by a pulsation is almost constant, the predetermined temperature range may be set (for example, the temperature, which has risen at the time of a previous pulsation, ±α) based on a rise degree of the body temperature which has risen at the time of the previous pulsation.

Referring back to FIG. 3, the extraction unit 31 extracts a change in temperature caused by a pulsation from the measurement result obtained by performing digital conversion by the conversion unit 33 and removing noise by the noise removal unit 34. More specifically, the extraction unit 31 executes peak detection processing for the measurement result, and extracts a timing at which the body temperature becomes highest.

Note that once a pulsebeat can be measured, it is possible to predict, based on the interval of the pulsebeat, a timing at which the body temperature becomes highest. The extraction unit 31 may stop extraction of a change in temperature in accordance with the interval of the measured pulsebeat for power saving. That is, the extraction unit 31 may intermittently extract a change in temperature. In this case, a period during which a change in temperature is extracted includes at least a period corresponding to one period of noise to be removed.

A measurement unit 32 mainly corresponds to the calculation unit 3d of FIG. 2, and operates as a pulsebeat measurement unit for calculating a pulsebeat based on the interval between changes in temperature extracted by the extraction unit 31. More specifically, the measurement unit 32 captures, as an R-R interval, a time interval between timings at which the body temperature becomes highest, and calculates a pulse rate based on the R-R interval.

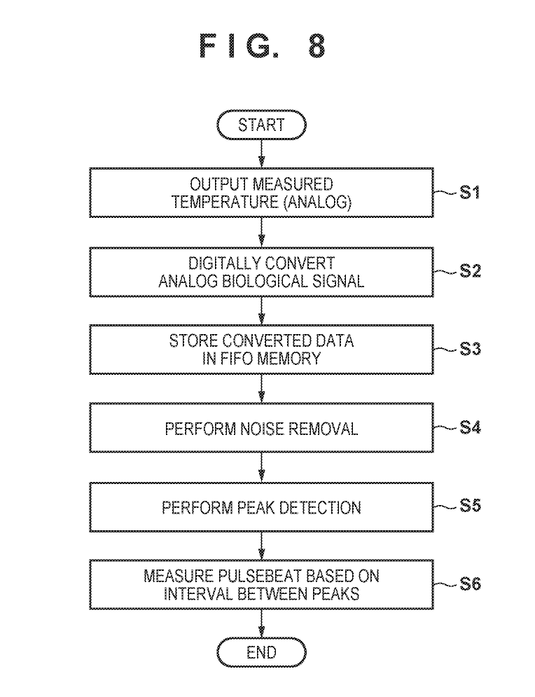

A pulsebeat measurement method in the pulsebeat measurement apparatus 1 will be described. FIG. 8 is a flowchart illustrating the pulsebeat measurement method. In step S1, the sensor unit 2 measures the temperature (body temperature) on the contact surface with the human body, and outputs the temperature to the amplifier 3a. Note that if no amplifier 3a is provided, the sensor unit 2 directly outputs the measured temperature to the A/D converter 3b. In step S2, after the amplifier 3a amplifies the temperature (analog biological signal) acquired from the sensor unit 2, the A/D converter 3b digitally converts the amplified analog biological signal at a sampling frequency corresponding to the frequency of noise to be removed. In step S3, the converted digital biological signals are stored in the FIFO memory 3c.

In step S4, the calculation unit 3d performs noise removal for the temperature measured by the sensor unit 2. More specifically, the calculation unit 3d removes the commercial power supply noise by calculating the moving average of the digital biological signals stored in the FIFO memory 3c, and simultaneously removes the aliasing by an LPF associated with the moving average. In step S5, the calculation unit 3d performs peak detection processing for the digital biological signals from which the noise has been removed, and extracts timings at which the temperature rises in association with a pulsation. After that, in step S6, the calculation unit 3d calculates a pulse rate based on the interval between the peaks of the temperature, thereby ending the process.

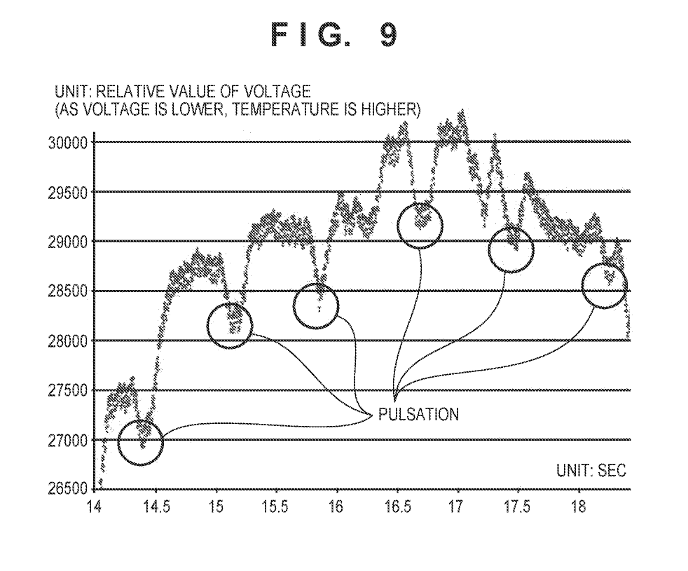

The result of an experiment performed by the present inventors will be described next. The present inventors measured the body temperature of the human body by a thermistor, and examined the relationship between a pulsebeat and the body temperature. FIG. 9 is a graph showing the relationship between the pulsebeat and body temperature of the human body. In FIG. 9, the ordinate represents the relative value of a voltage output from the thermistor and the abscissa represents the time. Note that as the voltage on the ordinate is lower, the temperature is higher.

As shown in FIG. 9, it has been found that the body temperature of the human body not only gradually changes in daily life but also instantaneously, slightly rises in association with a pulsation. Furthermore, since a small rise in temperature can be detected, as indicated by the experiment result, the pulsebeat of a subject can be measured based on the interval between the timings at which the temperature rises.

As described above, the pulsebeat measurement apparatus 1 measures the pulsebeat of the human body based on the interval between changes in temperature of the measurement portion with which the sensor unit 2 is in contact. Since a change in temperature can be detected near the artery, for example, it is only necessary for the pulsebeat measurement apparatus 1 to bring the sensor unit 2 into contact with a wrist, ankle, or the like. Therefore, the pulsebeat measurement apparatus 1 according to this embodiment does not restrain the action of the human body at all, and never gives a restraint feeling or oppressive feeling. In addition, since power required for temperature measurement is very small, a pulsebeat can be measured with very small power, as compared with the conventional photoplethysmography.

By calculating the moving average of the measurement results of the sensor unit 2 for one period of the frequency of the commercial power supply noise, the commercial power supply noise can be removed. Noise can be removed by excluding, from the processing target, a temperature falling outside the temperature range which the human body temperature can take, among the measurement results of the sensor unit 2. At this time, by setting, based on the body temperature of the subject measured by the sensor unit 2, the temperature range which the human body temperature can take, noise can be further reduced.

It can be expected to further reduce power consumption by intermittently extracting a change in temperature in accordance with the interval of the measured pulsebeat. That is, processing of extracting a change in temperature from the measurement result consumes predetermined power. Since this processing need only be performed in synchronism with the timing of a pulsation, it is possible to eliminate the processing during an unnecessary period by intermittently performing the processing based on the interval of the pulsation measured once. This can further reduce the power consumption. Note that since power required for temperature measurement by the sensor unit 2 is very small, temperature measurement by the sensor unit 2 may always or intermittently be performed.

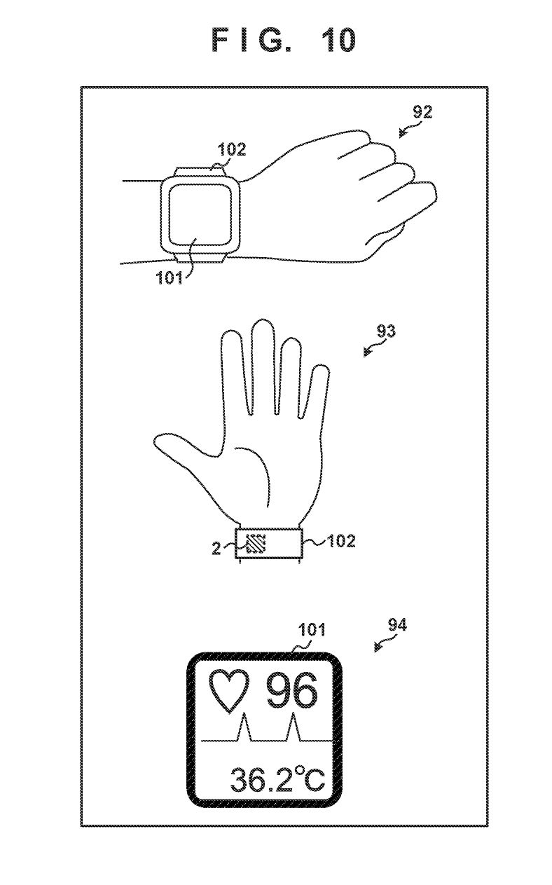

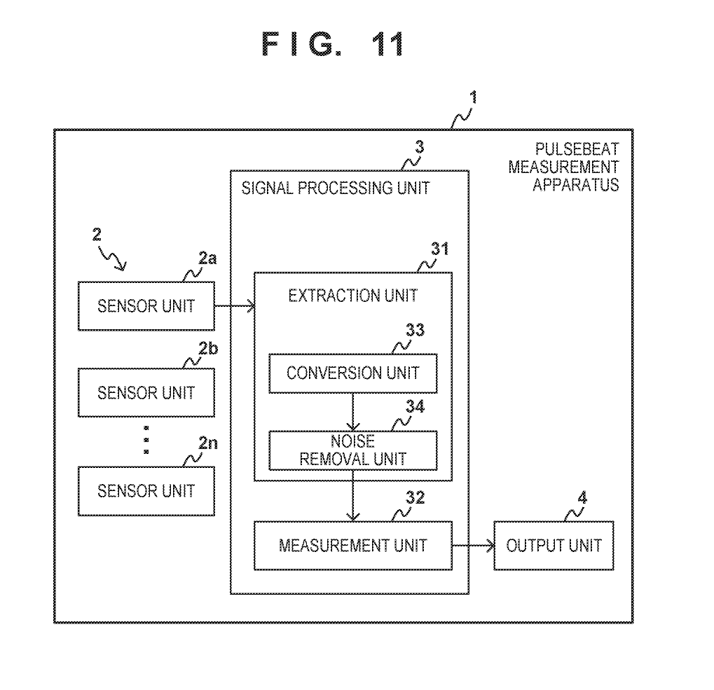

A wearable device including the pulsebeat measurement apparatus 1 according to this embodiment will be described. As denoted by reference numeral 92 in FIG. 10, the wearable device is a wristwatch type device worn around a wrist, and includes a display unit 101 on which a display screen and a touch panel are superimposed, and a belt 102 used to fix the wearable device around the wrist. As denoted by reference numeral 93 in FIG. 10, the sensor unit 2 is provided inside the belt 102. In the wearable device, the sensor unit 2 is in contact with the wrist (near the artery) of the user wearing the device, and measures the body temperature of the user. The wearable device monitors the body temperature measured by the sensor unit 2, and detects a small change in temperature caused by a pulsation, thereby measuring the pulsebeat of the user.

Note that the sensor unit 2 preferably reacts to only the body temperature of the user, and thus a heat insulation unit (not shown) for preventing the heat of the sensor unit 2 from being transferred from another device such as the display unit 101 is preferably provided. Similarly, a heat dissipation unit (not shown) for dissipating the heat of the sensor unit 2 which has been accumulated in association with a pulsation is preferably provided to keep the temperature of the sensor unit 2 almost constant before and after a pulsation. If, for example, the sensor unit 2 is arranged near the display unit 101 (watch) in the wristwatch type wearable device, a heat insulation unit may be provided between the display unit 101 and the sensor unit 2 (the heat dissipation unit, as needed).

The wearable device can notify the user wearing the device of his/her pulse rate by displaying the measured pulse rate on the display unit 101. Note that since the sensor unit 2 also measures the body temperature of the user, the display unit 101 can display not only the pulse rate but also vital data such as the body temperature. In this regard, in an example denoted by reference numeral 94, the pulse rate, pulse waveform, and current body temperature of the user are displayed on the display unit 101.

When the user wears the band, the above-described wearable device can readily acquire vital data such as a pulsebeat, so the action of the user is not restrained at all. Since power required for temperature measurement is very small, it is possible to measure a pulsebeat with low power consumption.

Note that the wristwatch type device has been exemplified as an example of the wearable device. The present invention, however, is not limited to this. The wearable device needs only contact a portion near the artery of the user, and may be a supporter or the like for protecting a neck, elbow, knee, ankle, or the like, or a spectacle type device. Note that if a spectacle type device is used, for example, the body temperature of the user can be measured by providing the sensor unit 2 in a modern portion of a spectacle frame which contacts a portion near the ear of the user, a temple portion of the spectacle frame which contacts a portion near the temple of the user, or the like.

In this embodiment, the FIFO memory 3c has been exemplified as an example of a method of implementing the noise removal unit 34. In this regard, the noise removal unit 34 need only remove noise such as commercial power supply noise and aliasing, and may perform noise removal by providing an arbitrary noise removal unit different from the FIFO memory 3c.

As noise to be removed, not only commercial power supply noise and aliasing but also various kinds of noise such as thermal noise (Johnson noise) are preferably removed together. For example, if thermal noise as random noise is removed, the thermal noise has no correlation. Thus, the noise removal unit 34 compares the measurement results of the sensor unit 2 along the time axis, extracts thermal noise based on the comparison result, and removes it. More specifically, the noise removal unit 34 divides the measurement results of the sensor unit 2 into a plurality of periods, and compares the comparison results. As a result, for example, if a signal within a predetermined frequency range appears only in the measurement result during a given period, this signal can be removed, thereby removing thermal noise. Note that the predetermined frequency range is a frequency range of a frequency necessary for pulsebeat detection or higher.

If, as a result of comparison, a signal within the predetermined frequency range commonly appears in the measurement results during two or more periods, the noise removal unit 34 can remove this signal, thereby removing thermal noise. Note that the predetermined frequency range is a frequency range of a frequency necessary for pulsebeat detection or higher.

### Second Embodiment

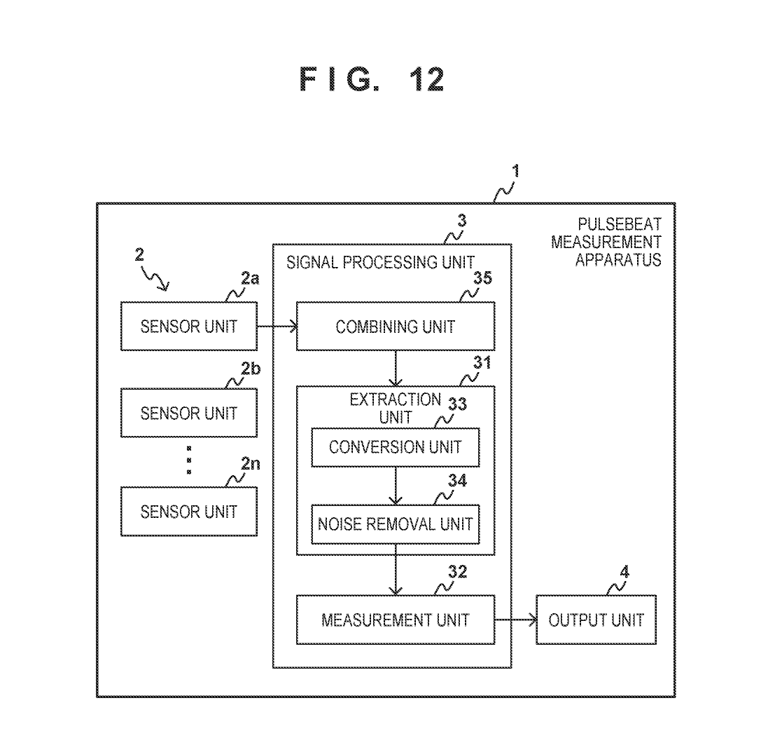

The second embodiment will be described next mainly concerning the difference from the first embodiment. FIG. 11 is a functional block diagram showing a pulsebeat measurement apparatus 1 according to this embodiment. Although the pulsebeat measurement apparatus 1 according to the first embodiment shown in FIG. 3 includes the one sensor unit 2, the pulsebeat measurement apparatus 1 according to this embodiment includes a plurality of sensor units 2a to 2n. Note that the sensor units 2a to 2n will be collectively referred to as sensor units 2 in the following explanation. In this embodiment, the sensor units 2 measure temperatures on a plurality of different contact surfaces of the same human body portion (blood vessel), respectively. More specifically, each of the sensor units 2a to 2n is provided in a portion where the pulsebeat measurement apparatus 1 is in contact with a measurement target blood vessel. At this time, to enable the measurement target blood vessel to be covered when a wearing state changes, the sensor units 2 can be configured to perform measurement at a plurality of positions in a direction orthogonal to the extension direction of the blood vessel.

In this embodiment, an extraction unit 31 executes peak detection processing for each of a plurality of measurement results acquired by the sensor units 2, and extracts a timing at which the body temperature becomes highest. At this time, the extraction unit 31 can improve the accuracy of pulsebeat measurement by comparing the plurality of measurement results acquired by the sensor units 2. For example, one of the sensor units 2 detects a rise in temperature although the plurality of sensor units 2 detect no rise in temperature, the extraction unit 31 can specify that the rise in temperature detected by the one sensor unit 2 is irrelevant to a pulsation, by comparing the measurement results. The accuracy of pulsebeat measurement is improved when the extraction unit 31 does not use, for timing extraction, the measurement result for which it has been specified that the temperature information is irrelevant to a pulsation.

The extraction unit 31 may extract a change in temperature based on the difference between the temperature measured by the sensor unit 2a and that measured by the sensor unit 2b. For example, assume that the sensor unit 2a is provided at a position where it is in contact with the inner side of a wrist, and the sensor unit 2b is provided at a position where it is in contact with the outer side of the wrist. In this case, since the sensor unit 2a is located near the blood vessel, the temperature readily changes in accordance with a pulsation. On the other hand, since the sensor unit 2b is far from the blood vessel, the temperature is difficult to change in accordance with a pulsation. It is possible to remove noise common to the sensor units 2a and 2b by subtracting the measurement result of the sensor unit 2b from that of the sensor unit 2a, and extract a rise in temperature caused by a pulsation detected by the sensor unit 2a, thereby improving the accuracy of pulsebeat measurement.

While the user wears the wearable device, the wearing state may change. If the wearing state changes, the contact states between the sensor units 2 and the user also change, so that the sensor unit 2 suitable for pulsebeat measurement is different before and after the change in wearing state. If the highest value of the body temperature detected by the sensor unit 2a is different from that of the body temperature detected by the sensor unit 2a at an immediately preceding timing, and is almost equal to that of the body temperature immediately precedingly detected by the sensor unit 2b, the extraction unit 31 determines that the wearing state of the pulsebeat measurement apparatus 1 has changed. In this case, the extraction unit 31 may extract, as a pulsation timing before the wearing state changes, the timing at which the sensor unit 2b immediately precedingly detects the highest value of the body temperature, and extract, as a pulsation timing after the wearing state changes, the timing at which the highest value of the body temperature detected by the sensor unit 2a is detected. This allows the pulsebeat measurement apparatus 1 to improve the accuracy of pulsebeat measurement when the wearing state changes.

In this embodiment, since the plurality of sensor units 2a to 2n are arranged near the measurement portion, the pulsebeat measurement apparatus 1 can improve the accuracy by obtaining the difference between the sensor units 2a to 2n, as compared with a case in which one sensor unit 2 is used to measure a pulsebeat. For example, even if pulse loss occurs in one sensor unit 2, another sensor unit 2 can support to accurately measure a pulsebeat, as compared with a case in which one sensor unit 2 is used to measure a pulsebeat. Furthermore, if the wearing state of the pulsebeat measurement apparatus 1 changes due to the operation of the wearer, the sensor unit 2 different from that before the wearing state changes measures the temperature of the measurement portion, thereby making it possible to measure a pulsebeat. As a result, the pulsebeat measurement apparatus 1 puts no heavy load on either the human body or the apparatus, and can continuously measure a pulsebeat even if the wearing state changes.

### Third Embodiment

The third embodiment will be described next mainly concerning the difference from the second embodiment. The pulsebeat measurement apparatus 1 according to the second embodiment digitally converts a plurality of analog biological signals respectively measured by the sensor units 2a to 2n, and performs noise removal for each signal. Therefore, the processing load unwantedly increases in accordance with the number of sensor units 2. To solve this problem, a pulsebeat measurement apparatus 1 according to this embodiment combines a plurality of analog biological signals respectively measured by a plurality of sensor units 2a to 2n, and performs digital conversion, noise removal, and the like for the combined analog biological signal, thereby reducing the processing load.

FIG. 12 is a functional block diagram showing the pulsebeat measurement apparatus 1 according to this embodiment. The difference from the pulsebeat measurement apparatus 1 according to the second embodiment shown in FIG. 11 is that a combining unit 35 for combining a plurality of analog biological signals from the sensor units 2 is provided.

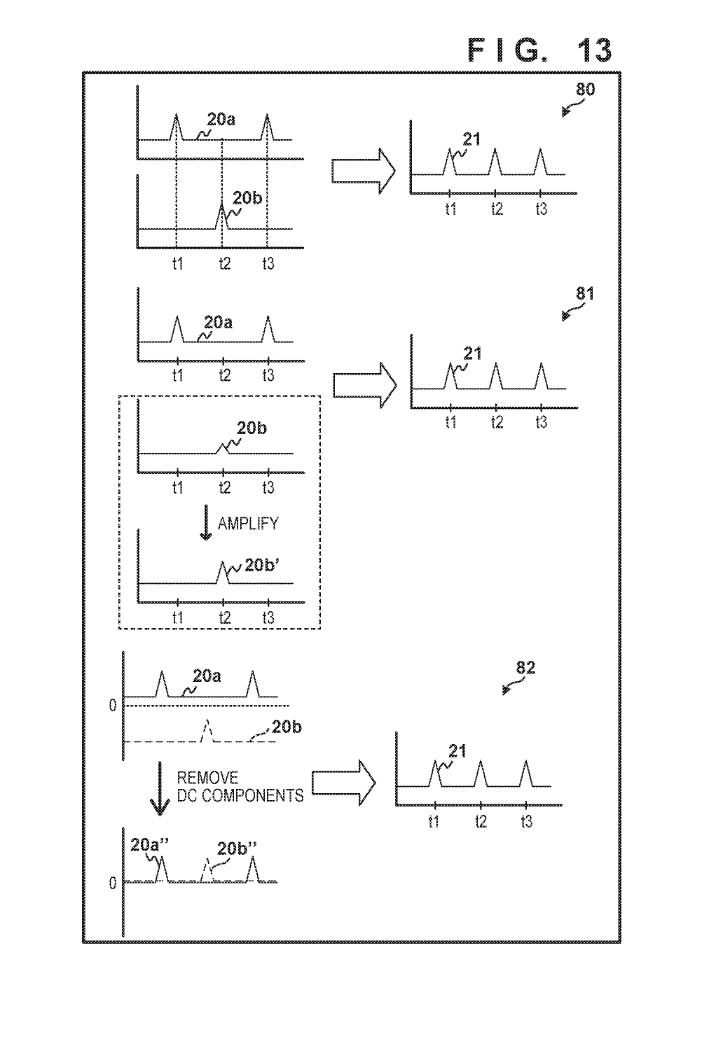

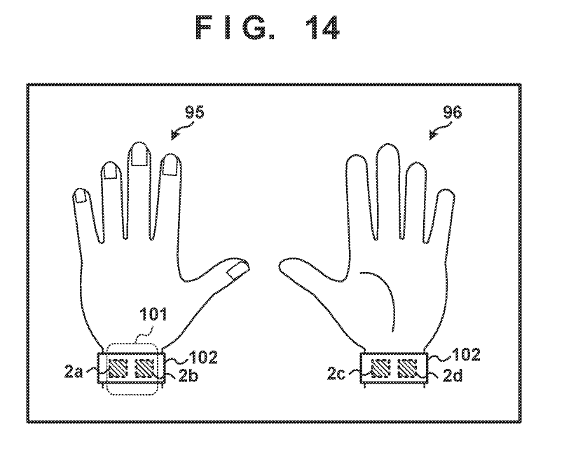

The combining unit 35 mainly corresponds to the amplifier 3a of FIG. 2, and combines the measurement results (analog data) of the sensor units 2. Combining of the measurement results by the combining unit 35 will now be described with reference to FIG. 13. Note that in FIG. 13, the measurement result of the sensor unit 2a is represented as a measurement result 20a, the measurement result of the sensor unit 2b different from the sensor unit 2a is represented as a measurement result 20b, and a result of combining the measurement results 20a and 20b is represented as a combined measurement result 21. Furthermore, for the sake of descriptive simplicity, FIG. 13 shows combining of only the two measurement results 20a and 20b, and illustrates no measurement results of the remaining sensor units 2. In FIG. 13, assume that the pulsation of a wearer occurs at timings t1, t2, and t3.

A wearable device can preferably measure a pulsebeat without making the wearer be conscious, and needs to be able to measure a correct pulsebeat even if a wearing state changes due to the operation of the wearer. As denoted by reference numeral 80 in FIG. 13, the sensor unit 2a detects a rise in temperature caused by the pulsation at each of timings t1 and t3, but cannot detect a rise in temperature caused by the pulsation at timing t2 due to a change in wearing state, as indicated by the measurement result 20a. On the other hand, as indicated by the measurement result 20b, the sensor unit 2b detects a rise in temperature caused by the pulsation at timing t2 when the sensor unit 2a cannot detect a rise in temperature caused by the pulsation due to a change in wearing state.

In this case, if a pulsebeat is measured only from the measurement result 20a of the sensor unit 2a, the period of the pulsebeat is unwantedly calculated to be longer than the actual period, like “t3−t1”. To solve this problem, the combining unit 35 combines the measurement result 20a of the sensor unit 2a and the measurement result 20b of the sensor unit 2b, thereby obtaining the combined measurement result 21. This can detect that the pulsation occurs at each of timings t1, t2, and t3, thereby measuring a correct pulsebeat. In addition, since it is only necessary to perform processing such as digital conversion only for the combined measurement result 21, it is possible to reduce the processing load.

If the measurement results are simply combined when the signal levels of the sensor units 2a to 2n are different, the measurement result of the sensor unit 2 with a low signal level is buried. In this regard, as denoted by reference numeral 81, although a rise in temperature caused by the pulsation can be detected in the measurement result 20b at timing t2 when a rise in temperature cannot be detected in the measurement result 20a, the signal level of the measurement result 20b is low. In this case, if the measurement results 20a and 20b are combined, the detection of the rise in temperature at timing t2 is buried, resulting in measurement of a wrong pulsebeat.

To solve this problem, the combining unit 35 amplifies the measurement result 20b whose signal level is equal to or lower than a predetermined level as a threshold, and then combines the measurement result 20a and an amplified measurement result 20b′. As a result, as denoted by reference numeral 81, the combined measurement result 21 in which the rise in temperature at timing t2 can be detected is obtained, thereby making it possible to measure a correct pulsebeat based on timings t1, t2, and t3 at which the temperature rises in association with the pulsation.

At this time, the combining unit 35 may weight a plurality of measurement results based on positions with which the sensor units 2a to 2n are in contact, and combine the plurality of weighted measurement results. For example, assume that the sensor unit 2a is provided at a position where it is in contact with the inner side of a wrist, and the sensor unit 2b is provided at a position where it is in contact with the outer side of the wrist. In this case, since the sensor unit 2a is closer to the blood vessel, it is considered that the reliability of the measurement result 20a of the sensor unit 2a is higher than that of the measurement result 20b of the sensor unit 2b. Thus, the combining unit 35 may multiply the measurement result 20a by a weighting factor larger than that for the measurement result 20b, and add a value obtained by the multiplication operation and the value of the measurement result 20b.

Depending on the performances of the sensor units 2a and 2b and situations around the sensor units 2a and 2b, DC components may be added to the measurement results, and the measurement results 20a and 20b may deviate from the zero point, as denoted by reference numeral 82. In this case, if the measurement results 20a and 20b are simply combined, a rise in temperature caused by a pulsation is buried, resulting in measurement of a wrong pulsebeat.

To solve this problem, the combining unit 35 removes (offsets) the DC components of the measurement results 20a and 20b, and then combines measurement results 20a″ and 20b″ after the removal of the DC components. As a result, the combined measurement result 21 in which a rise in temperature can be detected at each of timings t1, t2, and t3 can be obtained, thereby measuring a correct pulsebeat, as denoted by reference numeral 82.

As described above, in this embodiment, since a pulsebeat is measured from the combined measurement result obtained by combining the plurality of measurement results acquired by the sensor units 2, it is not necessary to perform processing such as digital conversion for each of the plurality of measurement results, and it is thus possible to reduce the processing load. In this case as well, even if the wearing state of the pulsebeat measurement apparatus 1 changes due to the operation of the wearer, another sensor unit 2 can detect a change in temperature of the measurement portion, thereby measuring a pulsebeat. As a result, the pulsebeat measurement apparatus 1 according to the third embodiment can measure a pulsebeat even if the wearing state changes while further reducing the load of the apparatus.

Note that in the second and third embodiments, the plurality of sensor units 2a to 2n are in contact with different positions of the same human body portion. However, the plurality of sensor units 2a to 2n may be in contact with different human body portions. For example, the sensor unit 2a may be in contact with the position of the blood vessel of a wrist and the sensor unit 2b may be in contact with the position of the blood vessel of an arm.

If the plurality of sensor units 2a to 2n are in contact with different human body portions, timings at which the sensor units 2 respectively detect the highest values of the body temperatures may shift from each other. The extraction unit 31 may correct the measurement result of the sensor unit 2a or 2b based on the difference between the timing at which the sensor unit 2a detects the highest value of the body temperature and the timing at which the sensor unit 2b detects the highest value of the body temperature.

If the pulsebeat measurement apparatus 1 includes the combining unit 35, as in the third embodiment, the combining unit 35 can generate a measurement result from which the influence of the timing shift has been removed, by combining the plurality of measurement results after the extraction unit 31 performs the above correction processing. Note that before the combining unit 35 combines the plurality of measurement results, the extraction unit 31 may amplify or attenuate the plurality of measurement results so that the magnitude of the maximum value of the plurality of measurement results falls within a predetermined range.

Note that if the plurality of sensor units 2a to 2n are provided at positions separated from each other, each of the plurality of sensor units 2a to 2n can be configured to wirelessly transmit the measurement result to a signal processing unit 3. For example, if the sensor unit 2a and the signal processing unit 3 are provided on a wrist, and the sensor unit 2b is provided on an arm, the sensor unit 2b may transmit the measurement result to the signal processing unit 3 using a wireless communication method capable of transmitting/receiving information at a short distance. Note that even if one sensor unit 2 is used, as in the first embodiment, the sensor unit 2 may be configured to wirelessly transmit the measurement result to the signal processing unit 3.

When the plurality of sensor units 2 are in contact with different human body portions, even if the wearing state of the sensor unit 2 in one human body portion is bad, the measurement result of the sensor unit 2 in another human body portion can be used. Therefore, even if there is a problem with the wearing state of the sensor unit 2 in one human body portion, it is possible to measure a pulsebeat.

An example of a wearable device including the pulsebeat measurement apparatus 1 with the plurality of sensor units 2a to 2n will be described next. Similarly to the first embodiment, the wearable device is a wristwatch type device worn around a wrist, and includes a display unit 101 on which a display screen and a touch panel are superimposed, and a belt 102 used to fix the wearable device around the wrist. However, as shown in FIG. 14, the plurality of sensor units 2a to 2d are provided inside the belt 102. The plurality of sensor units 2 are provided to reliably detect a change in temperature caused by a pulsation even if the wearing state of a wearable device 100 changes, and are arranged in a direction orthogonal to the extension direction of a measurement target blood vessel, as described above. In this example, the sensor units 2a and 2b are arranged on the side of the display unit 101 of the belt 102 (the outer side of the wrist), as denoted by reference numeral 95, and the sensor units 2c and 2d are arranged on the side facing the display unit 101 of the belt 102 (the inner side of the wrist), as denoted by reference numeral 96, thereby reliably measuring the temperature of the blood vessel of the wrist by one of the plurality of sensor units 2. Note that the actual number of sensor units 2 is not limited to four, and can be an arbitrary number of 2 or more. Referring to FIG. 14, the plurality of sensor units 2 are arranged in the direction orthogonal to the extension direction of the blood vessel. However, the plurality of sensor units 2 can also be arranged in the extension direction of the blood vessel.

Note that if thermal noise is superimposed on the measurement results of at least two or more sensor units 2 among the plurality of sensor units 2, the noise removal unit 34 can extract signals within a predetermined frequency range, which have correlation in at least two or more measurement results, based on a comparison result (in other words, a signal within a predetermined frequency range, which commonly appears in at least two or more measurement results). Then, the noise removal unit 34 can extract only the signals within the frequency range, which have correlation between the two or more sensor units 2, and remove them, thereby removing the thermal noise. Note that the predetermined frequency range indicates a frequency range of a frequency necessary for pulsebeat detection or higher.

For example, if thermal noise is superimposed on only the measurement result of one of the plurality of sensor units 2, a signal within a predetermined frequency range, which has no correlation with the measurement results of the remaining sensor units 2, may be extracted based on a comparison result. Then, the noise removal unit 34 can extract only a signal within the frequency range, and remove it, thereby removing the thermal noise.

The present invention is not limited to the above-described embodiments, and various changes and modifications can be made within the spirit and scope of the present invention. Therefore, to apprise the public of the scope of the present invention, the following claims are made.

1. A pulsebeat measurement apparatus comprising:
a temperature measurement unit configured to measure, by contacting a human body, a temperature of a contact surface; and
a processing unit configured to process a measurement result obtained by the temperature measurement unit,
wherein the processing unit includes:
an extraction unit configured to extract a change in temperature caused by a pulsation based on the measurement result, and
a pulsebeat measurement unit configured to measure a pulsebeat based on an interval between the changes in temperature.

2. The pulsebeat measurement apparatus according to claim 1, wherein
the extraction unit further includes a removal unit configured to perform noise removal for the measurement result by calculating a moving average of the measurement result at a period of AC noise to be removed, and
the extraction unit is further configured to extract the change in temperature from the measurement result after the noise removal by the removal unit.

3. The pulsebeat measurement apparatus according to claim 2,
wherein the removal unit is further configured to perform the noise removal for the measurement result by removing, from the measurement result, a temperature falling outside a temperature range which a human body temperature can take.

4. The pulsebeat measurement apparatus according to claim 3,
wherein the removal unit is further configured to set, as the temperature range which the human body temperature can take, a predetermined temperature range including the temperature measured by the temperature measurement unit.

5. The pulsebeat measurement apparatus according to claim 4,
wherein the predetermined temperature range is determined based on a change in temperature caused by an immediately preceding pulsation extracted by the extraction unit.

6. The pulsebeat measurement apparatus according to claim 1,
wherein the extraction unit is further configured to extract the change in temperature in accordance with a period of the pulsebeat measured by the pulsebeat measurement unit.

7. The pulsebeat measurement apparatus according to claim 1, further comprising a heat insulator configured to suppress transfer of heat between the processing unit and the temperature measurement unit.

8. The pulsebeat measurement apparatus according to claim 1, further comprising heat a dissipator configured to dissipate heat of the temperature measurement unit.

9. The pulsebeat measurement apparatus according to claim 1,
wherein the temperature measurement unit is further configured to measure a temperature on each of a plurality of contact surfaces.

10. The pulsebeat measurement apparatus according to claim 9, further comprising:
a combining unit configured to obtain a combined measurement result by combining a plurality of measurement results acquired by the temperature measurement unit on the plurality of contact surfaces,
wherein the extraction unit is further configured to extract the change in temperature based on the combined measurement result.

11. The pulsebeat measurement apparatus according to claim 10,
wherein the combining unit is further configured to amplify a measurement result, among the measurement results, whose level is lower than a threshold, and then combine the measurement results.

12. The pulsebeat measurement apparatus according to claim 10,
wherein the combining unit is further configured to weigh the measurement results based on the contact surfaces, and then combine the measurement results.

13. The pulsebeat measurement apparatus according to claim 9,
wherein the extraction unit is further configured to extract the change in temperature based on a difference between the plurality of measurement results acquired by the temperature measurement unit on the plurality of contact surfaces.

14. The pulsebeat measurement apparatus according to claim 1,
wherein the temperature measurement unit is further configured to wirelessly transmit the measurement result to the processing unit.

15. A wearable device comprising a pulsebeat measurement apparatus according to claim 1.

16. A pulsebeat measurement method of measuring a pulsebeat of a human body, comprising:
measuring a temperature of the human body;
extracting a change in temperature caused by a pulsation from the measured temperature; and
measuring the pulsebeat based on an interval between the extracted changes in temperature.
